(19) United States
(12) Patent Application Publication
(10) Pub. No.: US 2025/0207125 A1
(43) Pub. Date: Jun. 26, 2025

(54) VITRO TRANSCRIPTION OF SPATIALLY CAPTURED NUCLEIC ACIDS

(71) Applicant: 10x Genomics, Inc., Pleasanton, CA (US)

(72) Inventors: Anushka Gupta, Pleasanton, CA (US); Paul Eugene Lund, Pleasanton, CA (US)

(21) Appl. No.: 19/081,742

(22) Filed: Mar. 17, 2025

Related U.S. Application Data
(63) Continuation of application No. PCT/US2023/034788, filed on Oct. 10, 2023.
(60) Provisional application No. 63/414,704, filed on Oct. 10, 2022.

(51) Int. Cl. C12N 15/10; C12N 9/22; C12Q 1/6853

(52) U.S. Cl. CPC C12N 15/1096; C12N 9/22; C12Q 1/6853

(57) ABSTRACT

Provided herein are methods, compositions, and kits for in vitro transcription of spatially captured target nucleic acids on an array.

### CROSS-REFERENCE TO RELATED APPLICATIONS

Pursuant to 35 U.S.C. § 119 (e), this application is a continuation of International Application PCT/US2023/034788, with an international filing date of Oct. 10, 2023, which claims the benefit of priority to U.S. Provisional Patent Application No. 63/414,704, filed on Oct. 10, 2022, the contents of which are incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Sequencing methods from biological samples are inherently limited by the quantity of transcripts captured from a particular cell which can result in only a small fraction of the transcriptome captured from each cell. As such, when transcripts are captured from one cell, but not another, can result in an incomplete view of the biological activity of the biological sample. Methods to increase gene detection efficiency per cell result in reduced levels of undetected transcripts and a higher statistical power to parse out biological activity.

### SUMMARY

Typical spatial array workflows involve capturing transcripts on a substrate surface (e.g., a slide) with a capture probe including a spatial barcode and a capture domain, generating a second strand complementary to the capture probe, subsequently releasing the second strand, and optionally amplifying, prior to preparation of a sequencing library. An issue with typical spatial array workflows is the potential loss of transcripts during elution of second strand molecules after denaturation, which can result in reduced gene detection sensitivity. The present disclosure features methods and compositions for in vitro transcription of captured nucleic acids on the substrate prior to elution, thereby resulting in generation of multiple copies for each second strand molecule. The presence of a unique molecular identifier (UMI) allows for the reduction of transcript count to account for artifacts such as amplification bias.

Provided herein are methods for increasing the number of copies of a captured target nucleic acid from a biological sample on a spatial array, the method including: (a) providing a spatial array including a plurality of capture probes, where a capture probe of the plurality of capture probes includes: (i) a spatial barcode and (ii) a capture domain; (b) hybridizing a target nucleic acid from a biological sample to the capture domain of the capture probe, thereby generating a captured target nucleic acid and extending the capture probe using the captured target nucleic acid as a template, thereby generating an extended probe; (c) hybridizing an adaptor to the extended probe, where the adaptor includes a promoter sequence and extending the extended probe to include the adaptor, thereby generating an adaptor modified extended probe; and (d) contacting an RNA polymerase with the adaptor modified extended probe and in vitro transcribing one or more copies of the extended probe, thereby increasing the number of copies of the captured target nucleic acid on the spatial array relative to the number of copies generated in the absence of an RNA polymerase.

In some embodiments, the one or more copies of the adaptor modified extended probe are removed from the array.

In some embodiments, the method includes reverse transcribing the one or more copies of the adaptor modified extended probe thereby generating one or more RNA-cDNA hybrids. In some embodiments, the method includes removing the RNA from the one or more RNA-cDNA hybrids. In some embodiments, the removing includes the use of an RNase. In some embodiments, the RNase is RNase A, RNase C, RNase H, or RNase I. In some embodiments, the method includes generating a second strand DNA complementary to the cDNA.

In some embodiments, the method includes determining (i) the sequence of the spatial barcode or a complement thereof, and (ii) all or a portion of the sequence of the target nucleic acid, or a complement thereof, and using the determined sequences of (i) and (ii) to determine the location of the target nucleic acid in the biological sample. In some embodiments, the determining includes sequencing. In some embodiments, the sequencing includes high-throughput sequencing.

In some embodiments, the biological sample is disposed on the spatial array including the plurality of capture probes. In some embodiments, the biological sample is disposed on a substrate. In some embodiments, the method includes aligning the substrate with the biological sample with the spatial array, such that at least a portion of the biological sample is aligned with at least a portion of the spatial array.

In some embodiments, the method includes incorporating a polynucleotide sequence at 3′ end of the extended capture probe prior to step (c), where the polynucleotide sequence includes a heteropolynucleotide sequence. In some embodiments, the polynucleotide sequence includes a homopolynucleotide sequence. In some embodiments, incorporating the polynucleotide sequence includes the use of a reverse transcriptase. In some embodiments, incorporating the polynucleotide sequence includes the use of a terminal deoxynucleotidyl transferase.

Also provided herein are methods for generating copies of a captured target nucleic acid from a biological sample, the method including: (a) providing a spatial array including a plurality of capture probes, where a capture probe of the plurality of capture probes includes: (i) a spatial barcode and (ii) a capture domain; (b) hybridizing a target nucleic acid from a biological sample to the capture domain of the capture probe, thereby generating a captured target nucleic acid and extending the capture probe using the captured target nucleic acid as a template, thereby generating an extended probe; (c) hybridizing an adaptor to the extended probe, wherein the adaptor comprises (i) a sequence complementary to the extended probe and (ii) an RNA promoter sequence and extending the extended probe to include the adaptor, thereby generating a modified adaptor extended probe; (d) generating a second strand hybridized to the adaptor modified extended probe, where the second strand includes a sequence complementary to the spatial barcode and a nucleic acid sequence corresponding to the target nucleic acid; and (e) contacting an RNA polymerase with the spatial array, where the RNA polymerase in vitro transcribes one or more copies of the adaptor modified extended probe, thereby generating copies of the captured target nucleic acid on the spatial array.

In some embodiments, the one or more copies of the adaptor modified extended probe, or a complement thereof, are removed from the array.

In some embodiments, the method includes reverse transcribing the one or more copies of the modified adaptor extended probe, thereby generating one or more RNA-cDNA hybrids. In some embodiments, the method includes removing the RNA from the one or more RNA-cDNA hybrids. In some embodiments, the removing includes the use of an RNase, where the RNase includes one or more of RNase A, RNase C, RNase H, or RNase I. In some embodiments, the method includes removing the target nucleic acid from the modified adaptor extended probe after step (c), where the removing includes an RNase or denaturation.

In some embodiments, the method includes determining (i) the sequence of the spatial barcode or a complement thereof, and (ii) all or a portion of the sequence of the target nucleic acid, or a complement thereof, and using the determined sequences of (i) and (ii) to determine the location of the target nucleic acid in the biological sample. In some embodiments, the determining includes sequencing. In some embodiments, the sequencing includes high-throughput sequencing.

In some embodiments, the biological sample is disposed on the spatial array including the plurality of capture probes. In some embodiments, the biological sample is disposed on a substrate. In some embodiments, the method includes aligning the substrate with the biological sample with the spatial array, such that at least a portion of the biological sample is aligned with at least a portion of the spatial array.

In some embodiments, the method includes incorporating a polynucleotide sequence at the 3′ end of the extended probe prior to step (c), where the polynucleotide sequence in step (c) includes a heteropolynucleotide sequence. In some embodiments, the polynucleotide sequence includes a homopolynucleotide sequence. In some embodiments, incorporating the polynucleotide sequence in step (c) includes the use of a reverse transcriptase. In some embodiments, incorporating the polynucleotide sequence in step (c) includes the use of a terminal deoxynucleotidyl transferase.

Also provided herein are methods for amplifying captured target nucleic acids from a biological sample, the method including: (a) providing a spatial array including a plurality of capture probes, where a capture probe of the plurality of capture probes includes in a 5′ to 3′ direction: (i) a promoter sequence, (ii) a spatial barcode, and (iii) a capture domain; (b) hybridizing the target nucleic acids from the biological sample to the capture domains of the plurality of capture probes, thereby generating captured target nucleic acids; (c) extending the plurality of capture probes using the target nucleic acids as templates, thereby generating extended probes; and (d) contacting an RNA polymerase with the extended probes and transcribing one or more copies of the extended probes, thereby amplifying the captured target nucleic acids on the spatial array.

In some embodiments, the extended probes, or complements thereof, are removed from the array.

In some embodiments, the method includes reverse transcribing the amplified extended probes, thereby generating one or more RNA-cDNA hybrids. In some embodiments, the method includes removing the RNA from the one or more RNA-cDNA hybrids. In some embodiments, the removing includes the use of an RNase. In some embodiments, the RNase includes one or more of RNase A, RNase C, RNase H, or RNase I. In some embodiments, the method includes determining (i) the sequence of the spatial barcode or a complement thereof, and (ii) all or a portion of the sequence of the target nucleic acids, or a complement thereof, and using the determined sequences of (i) and (ii) to determine the location of the target nucleic acids in the biological sample. In some embodiments, the determining includes sequencing. In some embodiments, the sequencing includes high-throughput sequencing.

In some embodiments, the biological sample is disposed on the spatial array including the plurality of capture probes. In some embodiments, the biological sample is disposed on a substrate. In some embodiments, the method includes aligning the substrate with the biological sample with the spatial array, such that at least a portion of the biological sample is aligned with at least a portion of the spatial array.

In some embodiments, the method includes after step (c) incorporating a polynucleotide sequence including at least three nucleotides to the 3′ end of the extended probe. In some embodiments, the method includes hybridizing an adaptor to the extended probe, where the adaptor includes (i) a sequence complementary to the polynucleotide sequence and (ii) a primer sequence. In some embodiments, the polynucleotide sequence includes a heteropolynucleotide sequence. In some embodiments, the polynucleotide sequence includes a homopolynucleotide sequence. In some embodiments, incorporating the polynucleotide includes the use of a reverse transcriptase. In some embodiments, incorporating the polynucleotide sequence includes the use of a terminal deoxynucleotidyl transferase.

In some embodiments, the method includes staining the biological sample. In some embodiments, the method includes imaging the biological sample. In some embodiments, the staining includes hematoxylin and cosin staining. In some embodiments, the staining includes the use of a detectable label selected from the group consisting of a radioisotope, a fluorophore, a chemiluminescent compound, a bioluminescent compound, or a combination thereof.

In some embodiments, the plurality of capture probes includes a cleavage domain, one or more functional domains, a unique molecular identifier, and combinations thereof.

In some embodiments, the target nucleic acid is mRNA. In some embodiments, the target nucleic acid is DNA. In some embodiments, the method includes permeabilizing the biological sample. In some embodiments, the permeabilizing includes the use of a protease. In some embodiments, the protease includes pepsin. In some embodiments, the protease includes proteinase K.

In some embodiments, the spatial array includes one or more features. In some embodiments, the one or more features are selected from the group consisting of: a spot, an inkjet spot, a masked spot, a pit, a post, a well, a ridge, a divot, a hydrogel pad, and a bead.

In some embodiments, the method include migrating the target nucleic acid to the spatial array. In some embodiments, the migrating includes electrophoresis.

In some embodiments, the biological sample is a tissue sample. In some embodiments, the tissue sample is a fresh-frozen tissue sample. In some embodiments, the tissue sample is a fixed tissue sample. In some embodiments, the fixed tissue sample is a formalin-fixed paraffin-embedded tissue sample, an acetone fixed tissue sample, a methanol fixed tissue sample, or a paraformaldehyde tissue sample. In some embodiments, the biological sample is a tissue section. In some embodiments, the tissue section is a fresh-frozen tissue section. In some embodiments, the tissue section is a fixed tissue section. In some embodiments, the fixed tissue section is a formalin-fixed paraffin-embedded tissue section, an acetone fixed tissue section, a methanol fixed tissue section, or a paraformaldehyde tissue section.

In some embodiments, the promoter sequence includes one of a T7 RNA polymerase promoter sequence, a T3 RNA polymerase promoter sequence, or an SP6 RNA polymerase promoter sequence. In some embodiments, the polymerase includes one of a T7 RNA polymerase, a T3 RNA polymerase, or an SP6 RNA polymerase.

In some embodiments, the adaptor includes a template switch oligonucleotide sequence disposed between the sequence complementary to the polynucleotide sequence and the promoter sequence.

Also provided herein are kits including: (a) a spatial array including a plurality of capture probes, where a capture probe of the plurality of capture probes includes (i) a spatial barcode and (ii) a capture domain; (b) a plurality of adaptors, where an adaptor includes (i) a sequence complementary to a polynucleotide sequence added to an extended probe and (ii) a promoter sequence; and (c) a polymerase.

In some embodiments, the kit includes a reverse transcriptase.

In some embodiments, the promoter sequence includes one of a T7 RNA polymerase promoter sequence, a T3 RNA polymerase promoter sequence, or an SP6 RNA polymerase promoter sequence. In some embodiments, the polymerase includes one of a T7 RNA polymerase, a T3 RNA polymerase, or an SP6 RNA polymerase.

In some embodiments, the capture probe includes one or more functional domains, a unique molecular identifier, a cleavage domain, and combinations thereof.

In some embodiments, the kit includes a DNA polymerase.

In some embodiments, the kit includes one or more permeabilization reagents. In some embodiments, the one more permeabilization reagents include one or more of a protease, a lipase, a DNase, an RNase, a detergent, and combinations thereof. In some embodiments, the protease includes pepsin or proteinase K.

In some embodiments, the adaptor includes a template switch oligonucleotide sequence disposed between the polynucleotide sequence and the promoter sequence. In some embodiments, the kit includes instructions for performing any of the methods described herein.

Also provided herein are kits including: (a) a spatial array including a plurality of capture probes, where a capture probe of the plurality of capture probes includes in a 5′ to 3′ direction: (i) a promoter sequence, (ii) a spatial barcode, and (iii) a capture domain; (b) a plurality of adaptors, where an adaptor includes (i) a sequence complementary to a polynucleotide sequence added to an extended probe and (ii) a template switch oligonucleotide sequence; and (c) a polymerase.

In some embodiments, the kit includes a reverse transcriptase.

In some embodiments, the promoter sequence includes one of a T7 RNA polymerase promoter sequence, a T3 RNA polymerase promoter sequence, or an SP6 RNA polymerase promoter sequence. In some embodiments, the polymerase includes one of a T7 RNA polymerase, a T3 RNA polymerase, or an SP6 RNA polymerase.

In some embodiments, the capture probe includes one or more functional domains, a unique molecular identifier, a cleavage domain, and combinations thereof.

In some embodiments, the kit includes a DNA polymerase.

In some embodiments, the kit includes one or more permeabilization reagents. In some embodiments, the one more permeabilization reagents include one or more of a protease, a lipase, a DNase, an RNase, a detergent, and combinations thereof. In some embodiments, the protease includes pepsin or proteinase K.

In some embodiments, the adaptor includes a template switch oligonucleotide sequence disposed between the polynucleotide sequence and the promoter sequence.

In some embodiments, the kit includes instructions for performing any of the methods described herein.

Also provided herein are compositions including: (i) an extended probe including a spatial barcode, (ii) a sequence complementary to an analyte, and (iii) a polynucleotide sequence added to the 3′ end of the extended probe, and an adaptor including (i) a sequence hybridized to the polynucleotide sequence added to 3′ of the extended probe and (ii) a promoter sequence.

In some embodiments, the extended probe includes one or more functional domains, a cleavage domain, a unique molecular identifier, and combinations thereof.

In some embodiments, the composition includes a template switch oligonucleotide sequence disposed between the polynucleotide sequence added to the 3′ end of the extended probe and the promoter sequence.

In some embodiments, the promoter sequence includes one of a T7 RNA polymerase promoter sequence, a T3 RNA polymerase promoter sequence, or an SP6 RNA polymerase promoter sequence.

In some embodiments, the composition includes a reverse transcriptase. In some embodiments, the composition includes an RNA polymerase. In some embodiments, the RNA polymerase includes one of a T7 RNA polymerase, a T3 RNA polymerase, or an SP6 RNA polymerase.

In some embodiments, the polynucleotide sequence added to the 3′ end of the extended probe includes a heteropolynucleotide sequence. In some embodiments, the polynucleotide sequence added to the 3′ end of the extended probe includes a homopolynucleotide sequence.

In some embodiments, the composition includes one or more copies of the extended probe, or a complement thereof.

In some embodiments, the composition includes a second strand complementary to the extended probe.

In some embodiments, the composition includes one or more copies of the extended probe, or a complement thereof.

In some embodiments, the analyte includes mRNA. In some embodiments, the analyte includes DNA. In some embodiments, the composition includes a DNA polymerase.

Also provided herein are compositions including: a capture probe including in a 5′ to 3′ direction (i) an RNA polymerase promoter sequence, (ii) a spatial barcode, and (iii) a capture domain.

In some embodiments, the capture probe includes one or more functional domains, a cleavage domain, and a unique molecular identifier.

In some embodiments, the RNA polymerase promoter sequence is positioned 5′ to the unique molecular identifier. In some embodiments, the RNA polymerase promoter sequence includes one of a T7 RNA polymerase promoter sequence, a T3 RNA polymerase promoter sequence, or an SP6 RNA polymerase promoter sequence.

In some embodiments, the capture domain includes a homopolymeric sequence. In some embodiments, the homopolymeric sequence includes a poly (T) sequence. In some embodiments, the capture domain includes a gene-specific sequence. In some embodiments, the capture domain includes a random sequence.

Also provided herein are composition including: an extended probe including in a 5′ to 3′ direction: (i) an RNA polymerase promoter sequence, (ii) a spatial barcode, (iii) a sequence complementary to an analyte, and (iv) a polynucleotide sequence added to 3′ end of the extended probe, and an adaptor including a sequence hybridized to the polynucleotide sequence added to 3′ of the extended probe.

In some embodiments, the extended probe includes one or more functional domains, a cleavage domain, a unique molecular identifier, and combinations thereof.

In some embodiments, the adaptor includes a template switch oligonucleotide sequence.

In some embodiments, the RNA polymerase promoter sequence includes one of a T7 RNA polymerase promoter sequence, a T3 RNA polymerase promoter sequence, or an SP6 RNA polymerase promoter sequence.

In some embodiments, the composition includes a reverse transcriptase. In some embodiments, the composition includes an RNA polymerase. In some embodiments, the RNA polymerase includes one of a T7 RNA polymerase, a T3 RNA polymerase, or an SP6 RNA polymerase.

In some embodiments, the polynucleotide sequence added to the 3′ end of the extended probe includes a heteropolynucleotide sequence. In some embodiments, the polynucleotide sequence added to the 3′ end of the extended probe includes a homopolynucleotide sequence.

In some embodiments, the composition includes a second strand complementary to the extended probe.

In some embodiments, the composition includes one or more copies of the extended probe, or a complement thereof.

In some embodiments, the analyte is mRNA. In some embodiments, the analyte is DNA. In some embodiments, the composition includes a DNA polymerase.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram showing an exemplary in vitro transcription scheme to generate copies from a captured target nucleic acid on a spatial array.

FIG. 3 is a schematic diagram showing an exemplary in vitro transcription scheme to generate copies after second strand synthesis from a captured target nucleic acid on a spatial array.

FIG. 4 is a schematic diagram showing an exemplary in vitro transcription scheme to generate copies where a promoter sequence is engineered into the capture probe shown in FIG. 1.

FIGS. 5A-B are schematic diagrams depicting exemplary nucleic acid transfer embodiments. FIG. 5A shows an exemplary sandwiching process where a first substrate including a biological sample and a second substrate are brought into proximity with one another and a liquid reagent drop is introduced on the second substrate in proximity to the capture probes and in between the biological sample and the capture probes. FIG. 5B shows a fully formed sandwich configuration creating a chamber formed using one or more spacers, the first substrate, the second substrate including spatially barcoded capture probes, and the liquid reagent drop in contact with the capture probes and the biological sample.

### DETAILED DESCRIPTION

Typical spatial array workflows involve capturing transcripts, in some instances on a substrate surface (e.g., a slide) with a capture probe including a spatial barcode and a capture domain, generating a second strand complementary to the capture probe, subsequently releasing the second strand, and optionally amplifying prior to preparation of a sequencing library for determination of analyte expression profiles. An issue with typical spatial array workflows is the potential loss of transcripts during elution of second strand molecules after denaturation, which can result in reduced gene detection sensitivity. Amplification methods performed on the array could alleviate loss of rare transcripts, however, standard amplification techniques such as polymerase chain reaction (PCR) involve rapid thermal cycling, which can be incompatible with a substrate.

The present disclosure features methods and compositions for in vitro transcription and amplification of captured target nucleic acids on the spatial array prior to elution, thereby resulting in generation of multiple copies of the target nucleic acids or complements thereof. As disclosed herein, in vitro transcription can be performed by incorporating a promoter sequence either into the structure of the capture probe proper, or incorporating a promoter into the workflow at various points after capture and during processing of the target nucleic acid. The isothermal in vitro transcription and amplification strategies provided herein do not require thermal cycling and are capable of amplifying nucleic acids up to about 2 kilobases in length.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or cosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). Sec, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b) (viii) U.S. Patent Application Publication No. 2020/0277663.

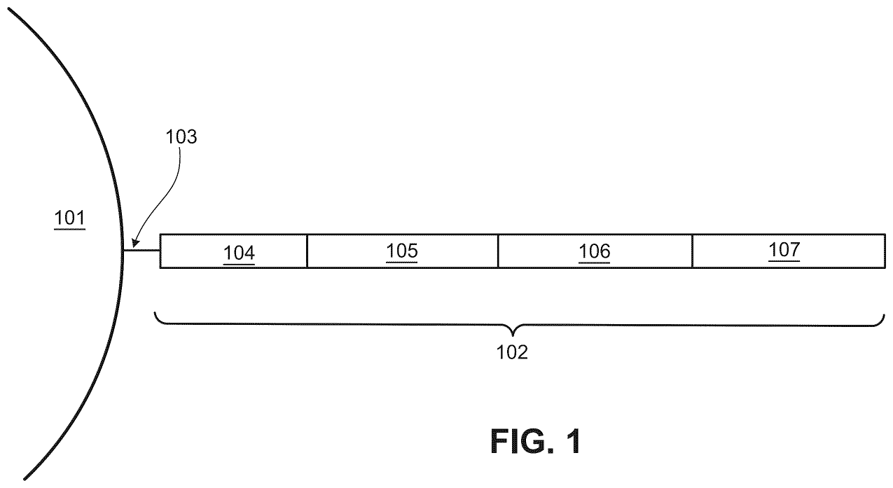

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. A functional sequence 104 could also include a promoter sequence, such as a promoter sequence useful for in vitro transcription methods as described herein. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature (e.g., a bead, a well, a spot on an array). In some embodiments, the UMI sequence 106 of a capture probe attached to a given feature is different from the UMI sequence of a different capture probe attached to the given feature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

As used herein, an “extended capture probe or extended probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

As used herein an “adaptor modified extended probe” refers to an extended capture probe or extended probe that is further extended. For example, an additional oligonucleotide can hybridize to the additional nucleotides added to the terminus of the extended capture probe as described herein. In some embodiments, the additional oligonucleotide is an adaptor having a nucleic acid sequence. In some embodiments, the adaptor includes a sequence complementary to the additional nucleotides added to the extended probe and a primer sequence (e.g., a template switch oligonucleotide sequence). In some embodiments, the adaptor further includes a promoter sequence. In such embodiments, the promoter sequence is 5′ to the primer sequence. After hybridization of the adaptor to the extended capture probe, the extended capture probe can be further extended to incorporate sequences complementary of the adaptor, thereby generating an adaptor modified extended probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. Sec, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c) (ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### In Vitro Transcription and Amplification of Nucleic Acids on a Spatial Array

The present disclosure features methods, compositions, and kits for in vitro transcription and amplification of captured target nucleic acids on a spatial array. The present disclosure describes methods of introducing a promoter sequence by various means to perform isothermal amplification of captured nucleic acids on an array. In some embodiments, the promoter sequence is incorporated into the capture probe. In some embodiments, the promoter sequence is incorporated into an extended probe thereby generating an adaptor modified extended probe. In some embodiments, the promoter sequence is incorporated into a second strand complementary to an adaptor modified extended probe. The presence of the promoter sequence enables isothermal amplification on the array to generate one or more copies of captured target nucleic acids or complements thereof. This amplification step reduces the loss of rare transcripts by generating one or more copies that can be further prepared in sequencing libraries.

Provided herein are methods for increasing the number of copies of a captured target nucleic acid from a biological sample on a spatial array, the method including: (a) providing a spatial array including a plurality of capture probes, where a capture probe of the plurality of capture probes includes: (i) a spatial barcode and (ii) a capture domain; (b) hybridizing a target nucleic acid from a biological sample to the capture domain of the capture probe, thereby generating a captured target nucleic acid and extending the capture probe using the captured target nucleic acid as a template, thereby generating an extended probe; (c) hybridizing an adaptor to the extended probe, where the adaptor includes a promoter sequence and extending the extended probe to include the adaptor, thereby generating an adaptor modified extended probe; and (d) contacting an RNA polymerase with the adaptor modified extended probe and in vitro transcribing one or more copies of the extended probe, thereby increasing the number of copies of the captured target nucleic acid on the spatial array relative to the number of copies generated in the absence of an RNA polymerase.

Also provided herein are methods for generating copies a captured target nucleic acid from a biological sample, the method including: (a) providing a spatial array including a plurality of capture probes, where a capture probe of the plurality of capture probes includes: (i) a spatial barcode and (ii) a capture domain; (b) hybridizing a target nucleic acid from a biological sample to the capture domain of the capture probe, thereby generating a captured target nucleic acid and extending the capture probe using the captured target nucleic acid as a template, thereby generating an extended probe; (c) hybridizing an adaptor to the extended probe, where the adaptor includes (i) a sequence complementary to the polynucleotide sequence of step (c) and (ii) an RNA promoter sequence and extending the extended probe to include the added polynucleotide sequence, thereby generating a modified adaptor extended probe; (d) generating a second strand hybridized to the adaptor modified extended probe, where the second strand includes a sequence complementary to the spatial barcode and a nucleic acid sequence corresponding to the target nucleic acid; and (c) contacting an RNA polymerase with the spatial array, where the RNA polymerase in vitro transcribes one or more copies of the adaptor modified extended probe, thereby generating copies of the captured target nucleic acid on the spatial array.

The present disclosure also features methods, compositions, and kits where a promoter sequence is included as part of the capture probe. For example, the promoter sequence can be located 5′ to the spatial barcode and the unique molecular identifier. Thus, provided herein are methods for amplifying captured target nucleic acids from a biological sample, the method including: (a) providing a spatial array including a plurality of capture probes, where a capture probe of the plurality of capture probes includes in a 5′ to 3′ direction: (i) a promoter sequence, (ii) a spatial barcode, and (iii) a capture domain; (b) hybridizing the target nucleic acids from the biological sample to the capture domains of the plurality of capture probes, thereby generating captured target nucleic acids; (c) extending the plurality of capture probes using the target nucleic acids as templates, thereby generating extended probes; and (d) contacting an RNA polymerase with the extended probes and transcribing one or more copies of the extended probes, thereby amplifying the captured target nucleic acids on the spatial array.

In some embodiments, the one or more copies of the adaptor modified extended probes are removed from the array. The one or more copies of the adaptor modified extended probes can be removed or eluted from the array by any suitable means including, but not limited to, aspirating, pipetting, etc.

In some embodiments, the method includes reverse transcribing the one or more copies of the adaptor modified extended probe, thereby generating one or more RNA-cDNA hybrids. The generated copies amplified on the array reduce the loss of low copy transcripts. In some embodiments, the RNA is removed from the RNA-cDNA hybrid. In some embodiments, the removing includes the use of an RNase. Any suitable RNase can be used, including but not limited to, RNase A, RNase C, RNase H, or RNase I.

In some embodiments, the methods include generating a second strand DNA that is complementary to the cDNA. In some embodiments, the methods include removing the target nucleic acid from the modified adaptor extended probe. In some embodiments, removing includes the use of an RNase (e.g., any RNase described herein). In some embodiments, removing includes denaturation (e.g., heat, KOH).

Non-limiting examples of target nucleic acids include nucleic acids such as DNA analytes or RNA analytes. Non-limiting examples of DNA analytes include genomic DNA, methylated DNA, specific methylated DNA sequences, fragmented DNA, mitochondrial DNA, in situ synthesized PCR products, and viral DNA.

Non-limiting examples of RNA analytes include various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

Target nucleic acids, such as mRNA, can be reverse transcribed after hybridizing to a capture domain (e.g., a poly (T) capture domain) to generate an extended probe e.g., RNA/cDNA. A polynucleotide sequence can be incorporated (e.g., added) to the end of the extended probe. For example, a reverse transcriptase or a terminal transferase can add a polynucleotide sequence in a template-independent manner (e.g., at least three non-templated nucleotides). In some embodiments, the polynucleotide sequence is a heteropolynucleotide sequence (e.g., CGC). In some embodiments, the polynucleotide sequence is a homopolynucleotide sequence (e.g., CCC).

In some embodiments, the capture domain is a homopolymeric sequence. In some embodiments, the homopolymeric sequence is a poly (T) sequence. In some embodiments, the capture domain is gene-specific. In some embodiments, the capture domain is a random sequence. In some embodiments, the capture domain is a degenerate sequence.

In some embodiments, such as when the promoter sequence is 5′ to the spatial barcode, after the polynucleotide sequence is added to the extended probe, an adaptor is hybridized to the extended probe, where the adaptor includes a sequence complementary to the polynucleotide sequence and a primer sequence (e.g., a template switch oligonucleotide sequence). In some embodiments, such as when the promoter sequence is 3′ to the spatial barcode, the adaptor includes a primer sequence (e.g., a template switch oligonucleotide sequence) disposed between the sequence complementary to the polynucleotide sequence and the promoter sequence.

In some embodiments, the array includes one or more features. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots). For example, the plurality of capture probes can be located on features on a substrate. In some embodiments, features include, but are not limited to, a spot, an inkjet spot, a masked spot, a pit, a post, a well, a ridge, a divot, a hydrogel pad, and a bead (e.g., a hydrogel bead).

In some embodiments, the biological sample can be stained. In some embodiments, the biological sample is stained after fixation. In some embodiments, the biological sample is stained before fixation. In some embodiments, the staining includes optical labels as described herein, including, but not limited to, fluorescent (e.g., fluorophore), radioactive (e.g., radioisotope), chemiluminescent (e.g., a chemiluminescent compound), a bioluminescent compound, calorimetric, or colorimetric detectable labels. In some embodiments, the staining includes a fluorescent antibody directed to a target nucleic acid (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and cosin (H&E) or periodic acid-schiff (PAS). In some embodiments, staining the biological sample includes the use of a biological stain including, but not limited to, acridine orange, Bismarck brown, carmine, coomassie blue, cresyl violet, DAPI, cosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can elapse between staining and/or imaging the biological sample.

In some embodiments, the biological sample is imaged. In some embodiments, the biological sample is imaged after fixation. In some embodiments, the biological sample is imaged before fixation. In some embodiments, imaging includes one or more of expansion microscopy, bright field microscopy, dark field microscopy, phase contrast microscopy, electron microscopy, fluorescence microscopy, reflection microscopy, interference microscopy and confocal microscopy.

In some embodiments, the biological sample is permeabilized. Permeabilization of a biological sample can occur on a substrate where the substrate is aligned with the array such that at least a portion of the biological sample is aligned with at least a portion of the array or directly on an array including a plurality of capture probes. In some embodiments, the biological sample is permeabilized with a protease. In some embodiments, the protease is one or more of pepsin, Proteinase K, and collagenase.

In some embodiments, the target nucleic acids that hybridize to the capture domains of the plurality of capture probes can migrate (e.g., diffuse) towards the capture probes through passive migration such as gravity. In some embodiments, the target nucleic acids that hybridize to the capture domains of the plurality of capture probes can migrate toward the capture probes through active migration. In some embodiments, the active migration includes electrophoresis.

In some embodiments, the biological sample is a tissue sample. In some embodiments, the tissue sample is a fixed tissue sample. For example, fixing the biological sample can include the use of a fixative including: ethanol, methanol, acetone, formaldehyde, paraformaldehyde-Triton, glutaraldehyde, and combinations thereof. In some embodiments, the fixed tissue sample is a formalin-fixed paraffin embedded tissue sample, paraformaldehyde fixed tissue sample, a methanol fixed tissue sample, or an acetone fixed tissue sample. In some embodiments, the tissue sample is a fresh-frozen tissue sample. In some embodiments, the biological sample is a tissue section. In some embodiments, tissue sample is a fresh-frozen tissue section. In some embodiments, the biological sample is a fixed tissue section (e.g., a fixed tissue section prepared by any of the methods described herein).

The methods described herein generate one or more copies of extended probes or adaptor modified extended probes through the use of a promoter sequence. Any suitable promoter sequence can be used including, but not limited to, a T7 RNA polymerase promoter sequence, a T3 RNA polymerase promoter sequence, or a SP6 RNA polymerase promoter sequence. In some embodiments, the methods include a T7 RNA polymerase, a T3 RNA polymerase, or a SP6 RNA polymerase.

In some embodiments, the methods described herein include determining (i) the sequence of the spatial barcode or a complement thereof, and (ii) all or a portion of the sequence of the target nucleic acid and using the determined sequences of (i) and (ii) to determine the location of the target nucleic acid in the biological sample. In some embodiments, the determining includes sequencing. In some embodiments, the sequencing includes high-throughput sequencing.

The resulting cDNA molecules generated from the captured target nucleic acids can be transferred (e.g., to a clean tube) for amplification, and/or library construction as described herein. The spatially-barcoded, full-length cDNA can be optionally further amplified via PCR prior to library construction. The cDNA products can then be enzymatically fragmented and size-selected in order to optimize the amplicon size. P5, P7, i7, and i5 can be incorporated into the library as for downstream sequencing, and additional library sequencing regions, such as TruSeq Read 2, can be added via End Repair, A-tailing, Adaptor Ligation, and PCR. The fragments can then be sequenced using paired-end sequencing using TruSeq Read 1 and TruSeq Read 2 as sequencing primer sites used in Illumina sequencing workflows. In some instances, the library is sequenced using any method described herein, such that different sequencing domains specific to other sequencing methods and techniques can be incorporated into a capture probe or introduced during library preparation. In some instances, the sequence of the target nucleic acids or proxies thereof is determined via sequencing. In some instances, the spatial barcode is sequenced, providing the location of the target nucleic acid.

Nucleic Acid Transfer or Sandwiching Configurations The present disclosure includes embodiments where a biological sample is disposed directly on a spatial array including a plurality of capture probes. However, also included in this disclosure are embodiments where the biological sample is disposed on a substrate (e.g., a slide) that is not spatially arrayed. In some embodiments, the biological sample can be aligned with a spatially arrayed substrate such that at least a portion of the biological sample is aligned with at least a portion of the spatial array (e.g., “sandwiched”).

In some embodiments, the target nucleic acid transfer sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the extension product(s) from the biological sample.

The target nucleic acid transfer sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

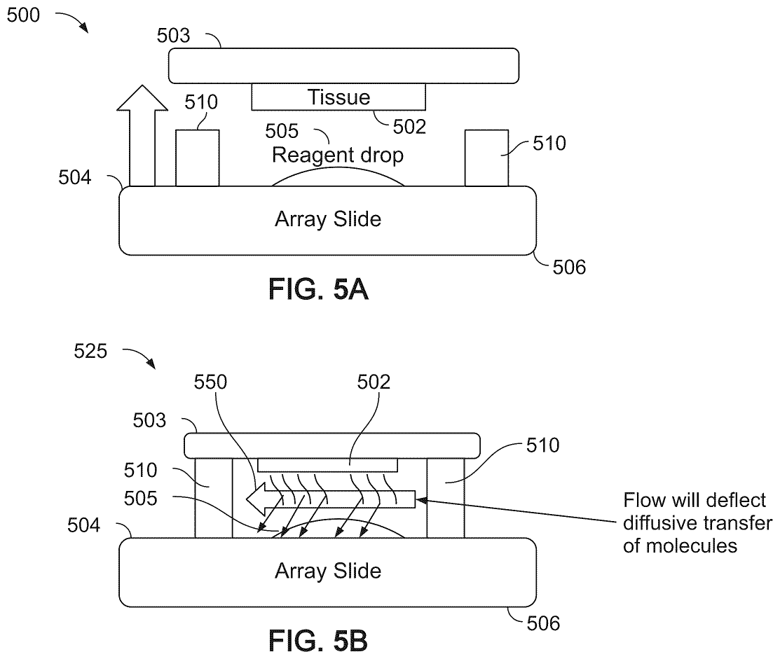

FIG. 5A shows an exemplary sandwiching process 500 where a first substrate (e.g., slide 503), including a biological sample 502 (e.g., a tissue section), and a second substrate (e.g., slide 504 including spatially barcoded capture probes 506) are brought into proximity with one another. As shown in FIG. 5A a liquid reagent drop (e.g., permeabilization solution 505) is introduced on the second substrate in proximity to the capture probes 506 and in between the biological sample 502 and the second substrate (e.g., slide 504 including spatially barcoded capture probes 506). The permeabilization solution 505 can release the target nucleic acids that can be captured (e.g., hybridized) by the capture probes 506 of the array. As further shown, one or more spacers 510 can be positioned between the first substrate (e.g., slide 503) and the second substrate (e.g., slide 504 including spatially barcoded capture probes 506). The one or more spacers 510 may be configured to maintain a separation distance between the first substrate and the second substrate. While the one or more spacers 510 is shown as disposed on the second substrate, the spacer may additionally or alternatively be disposed on the first substrate.

FIG. 5B shows a fully formed sandwich configuration creating a chamber 550 formed from the one or more spacers 510, the first substrate (e.g., the slide 503), and the second substrate (e.g., the slide 504 including spatially barcoded capture probes 506) in accordance with some example implementations. In FIG. 5B, the liquid reagent (e.g., the permeabilization solution 505) fills the volume of the chamber 550 and can create a permeabilization buffer that allows target nucleic acids to diffuse from the biological sample 502 toward the capture probes 506 of the second substrate (e.g., slide 504). In some aspects, flow of the permeabilization buffer may deflect the target nucleic acids from the biological sample 502 and can affect diffusive transfer of the target nucleic acids for spatial analysis. A partially or fully sealed chamber 550 resulting from the one or more spacers 510, the first substrate, and the second substrate can reduce or prevent flow from undesirable convective movement of transcripts and/or molecules over the diffusive transfer from the biological sample 502 to the capture probes 506.

### Compositions

The present disclosure also features compositions for amplifying a captured nucleic acid analyte on a spatial array. Thus, provided herein are compositions including an extended probe including (i) a spatial barcode, (ii) a sequence complementary to an analyte, and (iii) a polynucleotide sequence added to the 3′ end of the extended probe, and an adaptor including (i) a sequence hybridized to the polynucleotide sequence added to 3′ of the extended probe and (ii) a RNA polymerase promoter sequence.

Also provided herein are compositions including an extended probe that includes in a 5′ to 3′ direction (i) an RNA polymerase promoter sequence, (ii) a spatial barcode, (iii) a sequence complementary to an analyte, and (iv) a polynucleotide sequence added to 3′ end of the extended probe, and an adaptor including a sequence hybridized to the polynucleotide sequence added to 3′ of the extended probe.

In some embodiments, the extended probe includes one or more functional domains. In some embodiments, the extended probe includes a cleavage domain. In some embodiments, the extended probe includes a unique molecular identifier. In some embodiments, the extended probe includes one or more functional domains, a cleavage domain, a unique molecular identifier, and any combination thereof.

In some embodiments, the composition includes a template switch oligonucleotide sequence disposed between the polynucleotide sequence added to the 3′ end of the extended probe and the promoter sequence.

The compositions provided herein also include one or more enzymes. In some embodiments, the composition includes a reverse transcriptase. In some embodiments, the composition includes a DNA polymerase. In some embodiments, the composition includes an RNA polymerase. In some embodiments, the RNA polymerase is a T7 RNA polymerase. In some embodiments, the RNA polymerase is a T3 RNA polymerase. In some embodiments, the RNA polymerase is an SP6 RNA polymerase.

In some embodiments, the RNA polymerase promoter sequence is a T7 RNA polymerase promoter sequence. In some embodiments, the promoter sequence is a T3 RNA polymerase promoter sequence. In some embodiments, the promoter sequence is an SP6 RNA polymerase promoter sequence.

In some embodiments, the polynucleotide sequence added to the 3′ end of the extended probe comprises a heteropolynucleotide sequence. In some embodiments, the polynucleotide sequence added to the 3′ end of the extended probe comprises a homopolynucleotide sequence. For example, captured target nucleic acid analytes (e.g., RNA) can be reverse transcribed using the capture probe as a primer. An enzyme such as a reverse transcriptase or terminal transferase can add non-templated nucleotides to the 3′ end of the cDNA (e.g., an extended probe). For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA (e.g., an extended probe).

In some embodiments, the composition includes one or more copies of the extended probe, or a complement thereof.

In some embodiments, the composition includes a second strand complementary to the extended probe. In some embodiments, the composition includes one or more copies of the extended probe, or a complement thereof, generated from the second strand complementary to the extended probe.

In some embodiments, the target nucleic acid is RNA. Non-limiting examples RNA include various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

In some embodiments, the target nucleic acid is DNA. Non-limiting examples of DNA include genomic DNA, methylated DNA, specific methylated DNA sequences, fragmented DNA, mitochondrial DNA, in situ synthesized PCR products, and viral DNA.

In some embodiments, the composition includes a biological sample. In some embodiments, the biological sample is a tissue sample. In some embodiments, the tissue sample is a fresh-frozen tissue sample. In some embodiments, the tissue sample is a fixed tissue sample. In some embodiments, the fixed biological sample is a formalin-fixed paraffin-embedded tissue sample, a paraformaldehyde fixed tissue sample, an acetone fixed tissue sample, a methanol fixed tissue sample, or an ethanol fixed tissue sample. In some embodiments, the biological sample is a tissue section. In some embodiments, the tissue section is a fresh-frozen tissue section. In some embodiments, the tissue section is a fixed tissue section. In some embodiments, the fixed tissue section is a formalin-fixed paraffin-embedded tissue section, a paraformaldehyde fixed tissue section, an acetone fixed tissue section, a methanol fixed tissue section, or an ethanol fixed tissue section.

Also provided herein are compositions including a capture probe including in a 5′ to 3′ direction (i) an RNA polymerase promoter sequence, (ii) a spatial barcode, and (iii) a capture domain.

In some embodiments, the capture probe includes one or more functional domains, a cleavage domain, and a unique molecular identifier. In some embodiments, the RNA polymerase promoter sequence is positioned 5′ to the unique molecular identifier. In some embodiments, the RNA polymerase promoter sequence is a T7 RNA polymerase promoter sequence. In some embodiments, the RNA polymerase promoter sequence is a T3 RNA polymerase promoter sequence. In some embodiments, the RNA polymerase promoter sequence is an SP6 RNA polymerase promoter sequence.

In some embodiments, the capture domain is a homopolymeric sequence. In some embodiments, the homopolymeric sequence is a poly (T) sequence. In some embodiments, the capture domain is a gene-specific sequence. In some embodiments, the capture domain is a random sequence.

### Kits

The present disclosure also features kits for amplifying a captured nucleic acid on a spatial array. Thus, provided herein are kits including a spatial array including (a) a plurality of capture probes, where a capture probe of the plurality of capture probes includes (i) a spatial barcode and (ii) a capture domain; (b) a plurality of adaptors, where an adaptor includes (i) a sequence complementary to a polynucleotide sequence added to an extended probe and (ii) a promoter sequence; and a polymerase.

Also provided herein are kits including (a) a spatial array including a plurality of capture probes, where a capture probe of the plurality of capture probes includes in a 5′ to 3′ direction: (i) a promoter sequence, (ii) a spatial barcode, and (iii) a capture domain; (b) a plurality of adaptors, where an adaptor includes (i) a sequence complementary to a polynucleotide sequence added to an extended probe and (ii) a template switch oligonucleotide sequence; and (c) a polymerase.

The kits described herein can included one or more enzymes for amplifying captured nucleic acid analytes on a spatial array. In some embodiments, the kit includes a reverse transcriptase. In some embodiments, the kit includes a DNA polymerase.

In some embodiments, the polymerase is an RNA polymerase. Non-limiting examples of RNA polymerases include a T7 RNA polymerase, a T3 RNA polymerase, and an SP6 RNA polymerase. In some embodiments, the promoter sequence includes a sequence recognized by a polymerase (e.g., an RNA polymerase). In some embodiments, the promoter sequence is a T7 RNA polymerase promoter sequence. In some embodiments, the promoter sequence is a T3 RNA polymerase promoter sequence. In some embodiments, the promoter sequence is a SP6 RNA polymerase promoter sequence.

In some embodiments, the capture probe includes one or more functional domains (e.g., a primer binding site, a sequencing handle, etc.). In some embodiments, the capture probe includes a unique molecular identifier. In some embodiments, the capture probe includes a cleavage domain. In some embodiments, the capture probe includes one or more functional domains, a cleavage domain, a unique molecular identifier, or any combination thereof.

The kits described herein also include permeabilization reagents to permeabilize the biological sample such that nucleic acid analytes can diffuse or migrate (e.g., via electrophoresis) towards the spatial array. Thus, the kits described herein can include one or more permeabilization reagents where the permeabilization reagents include proteases, lipases, DNases, an RNase, a detergent, and combinations thereof. In some embodiments, the protease is pepsin. In some embodiments, the protease is proteinase K.

In some embodiments, the adaptor includes a template switch oligonucleotide sequence disposed between the polynucleotide sequence and the promoter sequence.

In some embodiments, the kit includes instructions for performing any of the methods of described herein.

### EXAMPLES

### Example 1. On-Slide In Vitro Transcription of a Captured Nucleic Acid

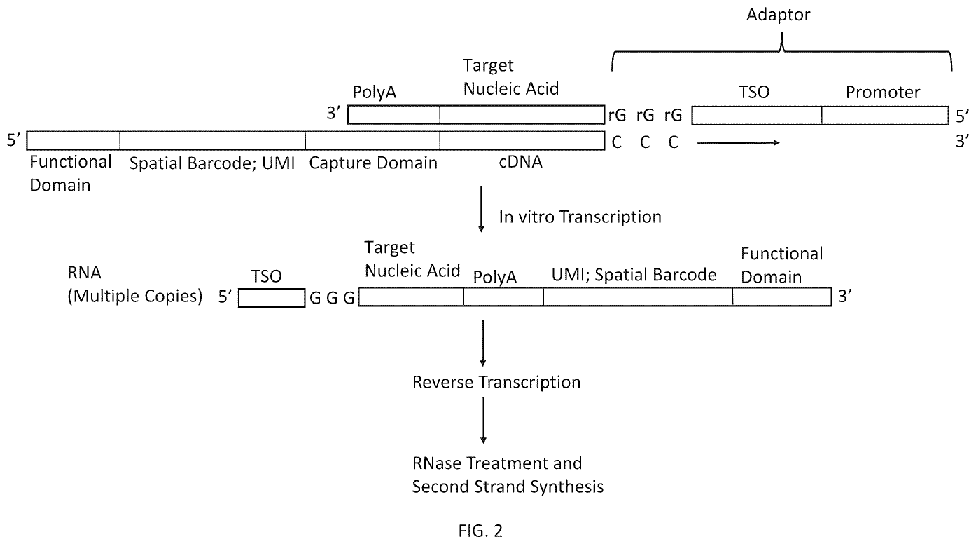

FIG. 1 is a schematic diagram showing an exemplary capture probe as described herein. The capture probe can include a cleavage domain, one or more functional sequences, a unique molecular identifier, a spatial barcode, and a capture domain. The capture domain facilitates capture of a target nucleic acid or a proxy of an analyte such as a ligation product or an analyte capture sequence of an analyte capture agent. FIG. 2 shows an exemplary method of in vitro transcribing and amplifying a captured nucleic acid on a spatial array that includes an exemplary capture probe including in a 5′ to 3′ direction a functional domain, a spatial barcode, a unique molecular identifier, and a capture domain. FIG. 2 shows capture of a target nucleic acid (e.g., mRNA), however, while the target nucleic acid in FIG. 2 is polyadenylated it is appreciated that other target nucleic acids can be captured by a capture probe when the capture domain comprises a sequence that is complementary to sequences in a target nucleic acid other than polyA. FIG. 2 shows reverse transcription to generate a cDNA complementary to the captured target nucleic acid, followed by an added polynucleotide sequence (e.g., CCC). Again, the polynucleotide sequence added to the extended probe can be either a heteropolynucleotide sequence or a homopolynucleotide sequence. An adaptor is hybridized to the polynucleotide sequence, the adaptor includes a primer sequence (e.g., a template switch oligonucleotide sequence) and a promoter sequence for in vitro transcription (e.g., any of the promoter sequences described herein). The extended probe can be further extended to include the sequences from the adaptor, thereby generating an adaptor modified extended probe.

In vitro transcription with an RNA polymerase (e.g., any of the RNA polymerases described herein) produces multiple RNA copies as shown in FIG. 2, which are subsequently collected from the array, reversed transcribed, and treated with an RNase (e.g., to remove the RNA from the cDNA/RNA hybrid). Optionally, second strand synthesis is performed before preparation of a sequencing library. Generating multiple copies on the array reduces the loss of rare transcripts during downstream steps and preparation.

### Example 2. On-Slide In Vitro Transcription of a Captured Nucleic Acid Via a Second Strand

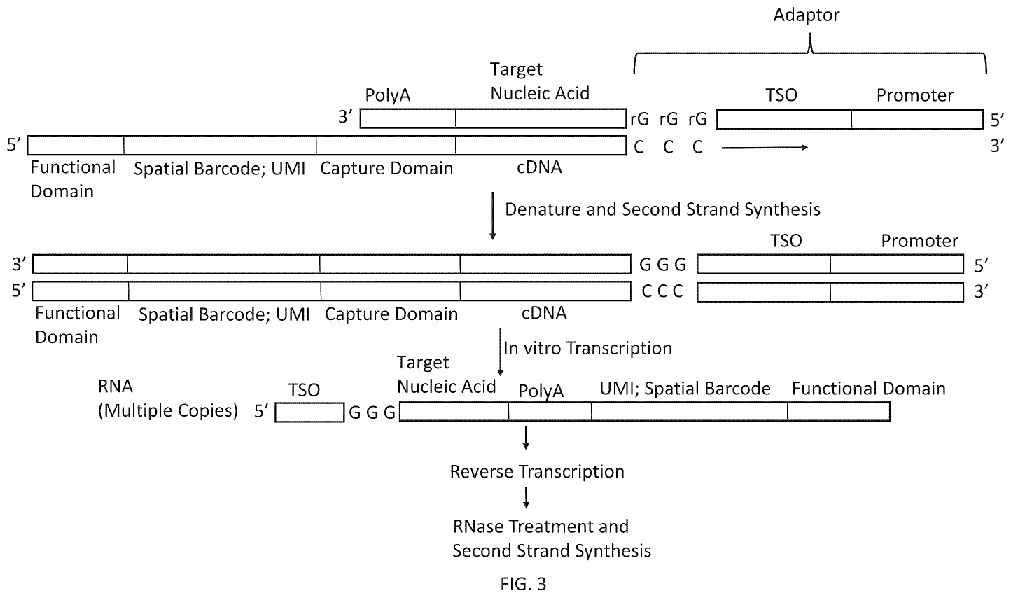

FIG. 3 is a variation of the method shown in FIG. 2 with the same exemplary capture probe and methods that include hybridization of the target nucleic acid, reverse transcription to generate a cDNA complementary to the captured target nucleic acid, addition of the polynucleotide sequence, hybridization of the adaptor, and extension of the extended probe to generate an adaptor modified extended probe. However, after generation of the adaptor modified extended probe, the target nucleic acid and the adaptor are removed (e.g., denatured) from the adaptor modified extended probe and second strand synthesis is performed to generate a double-stranded molecule on the array.

Next, in vitro transcription with an RNA polymerase (e.g., any of the RNA polymerases described herein) produces multiple RNA copies as shown in FIG. 3, which are collected from the array, reversed transcribed, and treated with an RNase (e.g., to remove the RNA from the cDNA/RNA hybrid). Optionally, second strand synthesis is performed before preparation of a sequencing library. Generating multiple copies on the array reduces the loss of rare transcripts during downstream steps and preparation.

### Example 3. On-Slide In Vitro Transcription of a Captured Nucleic Acid with a Capture Probe Including a Promoter Sequence

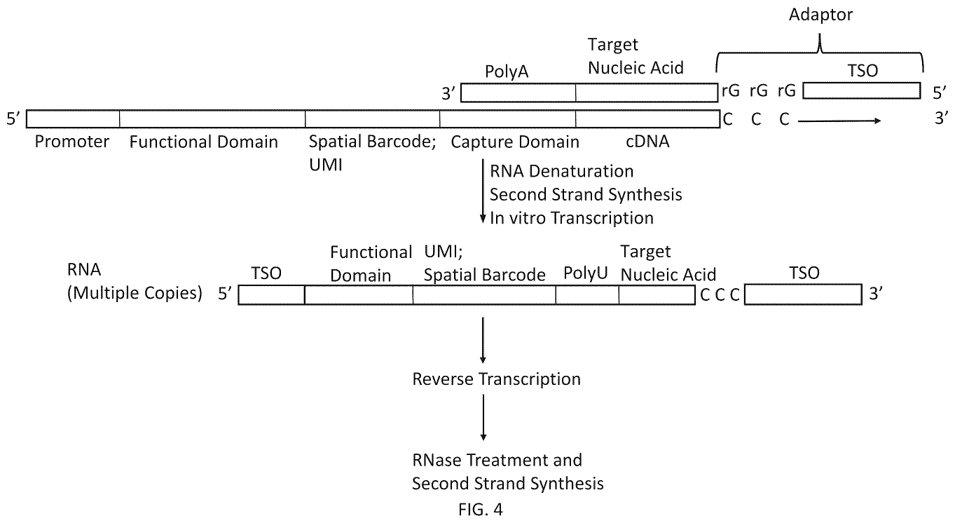

FIG. 4 shows an exemplary capture probe that also includes a promoter sequence, such as an RNA promoter sequence, included in the structure of the capture probe affixed to the spatial array. FIG. 4 shows the promoter sequence at the 5′ end of the capture probe. The steps of hybridization of the target nucleic acid, reverse transcription to generate a cDNA complementary to the captured target nucleic acid, addition of a polynucleotide sequence, hybridization of the adaptor, and extension are performed similarly as described in Example 1. In this example the adaptor includes a sequence complementary to the added polynucleotide sequence and a primer sequence (e.g., a template switch oligonucleotide sequence), but does not include a promoter sequence since it is present at the 5′ end of the capture probe.

After generation of the extended probe (e.g., cDNA), the target nucleic acid and the adaptor are removed (e.g., denatured) from the extended probe and second strand synthesis is performed to generate a double-stranded molecule on the array.

In vitro transcription with an RNA polymerase (e.g., any of the RNA polymerases described herein) produces multiple RNA copies as shown in FIG. 4, which are then collected from the array, reversed transcribed, and treated with an RNase (e.g., to remove the RNA from the cDNA/RNA hybrid). Optionally, second strand synthesis is performed before preparation of a sequencing library. Generating multiple copies on the array reduces the loss of rare transcripts during downstream steps and preparation.

What is claimed is:

1. A method for increasing the number of copies of a captured target nucleic acid from a biological sample on a spatial array, the method comprising:
(a) providing the spatial array comprising a plurality of capture probes, wherein a capture probe of the plurality of capture probes comprises: (i) a spatial barcode and (ii) a capture domain;
(b) hybridizing a target nucleic acid from the biological sample to the capture domain of the capture probe, thereby generating the captured target nucleic acid and extending the capture probe using the captured target nucleic acid as a template, thereby generating an extended probe;
(c) hybridizing an adaptor to the extended probe, wherein the adaptor comprises a promoter sequence and extending the extended probe using the adaptor as a template, thereby generating an adaptor modified extended probe; and
(d) contacting an RNA polymerase with the adaptor modified extended probe and in vitro transcribing one or more copies of the adaptor modified extended probe, thereby increasing the number of copies of the captured target nucleic acid on the spatial array relative to the number of copies generated in the absence of the RNA polymerase.

2. The method of claim 1, wherein the one or more copies of the adaptor modified extended probe are removed from the spatial array.

3. The method of claim 1, further comprising reverse transcribing the one or more copies of the adaptor modified extended probe, thereby generating one or more RNA-cDNA hybrids.

4. The method of claim 3, further comprising removing RNA from the one or more RNA-cDNA hybrids.

5. The method of claim 4, wherein the removing comprises an RNase.

6. The method of claim 5, further comprising generating a second strand DNA complementary to the cDNA, wherein the second strand comprises a sequence complementary to the spatial barcode and a nucleic acid sequence corresponding to the target nucleic acid.

7. The method of claim 1, further comprising determining (i) the sequence of the spatial barcode or a complement thereof, and (ii) all or a portion of the sequence of the target nucleic acid or a complement thereof.

8. The method of claim 7, further comprising using the determined sequences of (i) and (ii) to determine the location of the target nucleic acid in the biological sample.

9. The method of claim 1, wherein the biological sample is disposed on the spatial array comprising the plurality of capture probes or wherein the biological sample is disposed on a substrate.

10. The method of claim 9, further comprising aligning the substrate with the biological sample with the spatial array, such that at least a portion of the biological sample is aligned with at least a portion of the spatial array.

11. The method of claim 1, further comprising incorporating a polynucleotide sequence at the 3′ end of the extended probe prior to (c).

12. The method of claim 11, wherein incorporating the polynucleotide sequence comprises a reverse transcriptase.

13. The method of claim 1, further comprising staining the biological sample and/or imaging the biological sample.

14. The method of claim 1, wherein the plurality of capture probes comprises a cleavage domain, one or more functional domains, a unique molecular identifier, or a combination thereof.

15. The method of claim 1, wherein the target nucleic acid is mRNA.

16. The method of claim 1, wherein the target nucleic acid is DNA.

17. The method of claim 1, further comprising permeabilizing the biological sample.

18. The method of claim 1, wherein the spatial array comprises one or more features.

19. The method of claim 1, wherein the biological sample is a tissue sample.

20. The method of claim 1, wherein the RNA polymerase comprises a T7 RNA polymerase, a T3 RNA polymerase, or an SP6 RNA polymerase.
